(19) United States
(12) Patent Application Publication
(10) Pub. No.: US 2018/0110743 A1
(43) Pub. Date: Apr. 26, 2018

(54) COMPOSITIONS AND METHODS FOR TREATING HEPATOCELLULAR CARCINOMA

(71) Applicants: Northwestern University, Evanston, IL (US); Zhejiang University, Hangzhou, Zhejiang Province (CN)

(72) Inventors: Sui Huang, Chicago, IL (US); Zhi Chen, Hangzhou (CN); Yanning Liu, Hangzhou (CN); John T. Norton, Carol Stream, IL (US); Chen Wang, Chicago, IL (US); Guohua Lou, Hangzhou (CN); Richard M. Green, River Forest, IL (US)

(73) Assignee: Northwestern University, Evanston, IL (US)

(21) Appl. No.: 15/559,327

(22) PCT Filed: Mar. 18, 2016
(86) PCT No.: PCT/US2016/023212

§ 371 Date: Sep. 18, 2017

Related U.S. Application Data
(60) Provisional application No. 62/135,435, filed on Mar. 19, 2015.

(51) Int. Cl. A61K 31/137; A61P 35/00; A61K 31/44

(52) U.S. Cl. CPC A61K 31/137; A61K 31/44; A61P 35/00

(57) ABSTRACT

Provided herein are compositions and methods for treating, ameliorating, or preventing hepatocellular carcinoma in patients. In particular, the invention relates to methods of treating, ameliorating, or preventing hepatocellular carcinoma in a patient, comprising administering 6-methoxyethylamino-numonafide alone or in combination with sorafenib.

### CROSS REFERENCE TO RELATED APPLICATIONS

The present invention claims the priority benefit of U.S. Provisional Patent Application 62/135,435, filed Mar. 19, 2015, which is incorporated by reference in its entirety.

### STATEMENT REGARDING FEDERALLY SPONSORED RESEARCH OR DEVELOPMENT

This invention was made with government support under grand number GM078555 awarded by the National Institutes of Health. The government has certain rights in the invention.

### FIELD OF THE INVENTION

Provided herein are compositions and methods for treating, ameliorating, or preventing hepatocellular carcinoma in patients. In particular, the invention relates to methods of treating, ameliorating, or preventing hepatocellular carcinoma in a patient, comprising administering to the patient 6-methoxyethylamino-numonafide alone or in combination with sorafenib.

### BACKGROUND OF THE INVENTION

Hepatocellular carcinoma (HCC) is one of the most common causes of cancer deaths worldwide. In 2008, an estimated 750,000 new cases of liver cancer occurred and approximately 700,000 people died of this cancer worldwide; an increase from 626,000 new liver cancers and 600,000 deaths from liver cancer in 2002¹. While HCC was previously a public health problem limited primarily to Asia and Africa, the incidence of HCC in the United States is rapidly rising and will likely continue to rise for several decades. The age-adjusted incidence of HCC has tripled in the United States between 1975 and 2014, rising from 1.6 to 4.9 per 100,000 people². Last accessed May 21, 2014, HCC is now the ninth leading cause of cancer deaths in the United States and the third leading cause of cancer deaths worldwide³⁻⁴. In the US, the incidence of HCC is rising predominantly due to the endemic of hepatitis C. Once cirrhosis occurs in patients with hepatitis C, HCC develops at an annual rate of 1-5%⁵⁻⁷. In addition, the epidemic of obesity in America is resulting in a rapid rise in the incidence of nonalcoholic steatohepatitis (NASH)/cryptogenic cirrhosis which is also a major risk factor for the development of HCC⁸⁻⁹. Therefore, HCC in the United States is rapidly becoming a major health problem.

Improved methods for treating hepatocellular carcinoma are needed.

### SUMMARY

Hepatocellular carcinoma (HCC) is the third leading form of cancer worldwide and the incidence is increasing rapidly in the United States, tripling over the past 3 decades. Unfortunately, chemotherapeutic treatment strategies against localized and metastatic HCC are ineffective, leading to a high mortality from the disease. Sorafenib is the sole FDA approved chemotherapeutic currently used clinically for the disease and it shows limited efficacy with substantial toxicities. A novel small molecule, 6-methoxyethylamino-numonafide (MEAN), shows promise in treating HCC xenografts in vivo. MEAN is highly effective against two murine xenograft models of human HCC cell lines (Huh7 and HepG2).

Experiments conducted during the course of preparing embodiments for the invention determined that at the same concentration and treatment strategies, MEAN is more efficacious and less toxic than sorafenib. Treatment by MEAN at an efficacious dose was shown to not significantly impact animal body weight. Sorafenib in combination with MEAN was shown to inhibit tumor growth to a greater extent than single agent treatments and adding MEAN was shown to not significantly increase toxicities compared to sorafenib alone. Mechanistically, MEAN was shown to suppress c-myc expression and increase expression of several tumor suppressors, including SHP-1 and TXNIP. MEAN was shown to effectively inhibit cancer cell growth in several drug resistant cell lines with activated P-glycoprotein drug efflux pumps and was shown to have a drug like single dose pharmacokinetic profile. Altogether, these experiments demonstrate that MEAN is effective against HCC tumor growth as monotherapy and in combination with sorafenib, and is an excellent candidate for clinical development as a therapeutic agent for HCC management.

Accordingly, provided herein are compositions and methods for treating, ameliorating, or preventing hepatocellular carcinoma in patients. In particular, the invention relates to methods of treating, ameliorating, or preventing hepatocellular carcinoma in a patient, comprising administering to the patient 6-methoxyethylamino-numonafide alone or in combination with sorafenib.

In certain embodiments, the present invention provides methods of treating, ameliorating, or preventing hepatocellular carcinoma in a patient comprising administering to said patient a therapeutically effective amount of 6-methoxyethylamino-numonafide (MEAN):

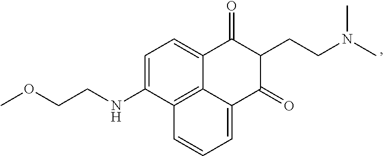

including pharmaceutically acceptable salts, solvates, and/or prodrugs thereof.

In some embodiments, the administering results in decreased c-myc expression. In some embodiments, the administering results in increased SHP-1 expression. In some embodiments, the administering results in increased TXNIP expression.

In some embodiments, the method further comprises co-administration of a therapeutically effective amount of sorafenib

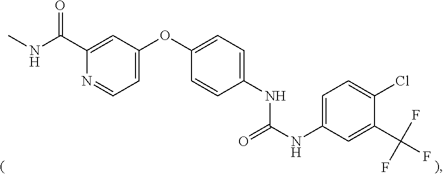 [SMILES: CNC(=O)c1cc(Oc2ccc(NC(=O)Nc3ccc(Cl)c(C(F)(F)F)c3)cc2)ccn1]

including pharmaceutically acceptable salts, solvates, and/or prodrugs thereof.

In some embodiments, the patient is a human patient.

In certain embodiments, the present invention provides methods of treating, ameliorating, or preventing hepatocellular carcinoma in a patient comprising administering to said patient a therapeutically effective amount of a pharmaceutical composition comprising a therapeutically effective amount of 6-methoxyethylamino-numonafide (MEAN):

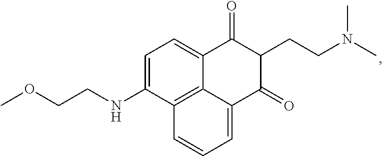

including pharmaceutically acceptable salts, solvates, and/or prodrugs thereof.

In some embodiments, the administering results in decreased c-myc expression. In some embodiments, the administering results in increased SHP-1 expression. In some embodiments, the administering results in increased TXNIP expression.

In some embodiments, the patient is a human patient.

In some embodiments, the methods further comprise co-administration of a pharmaceutical composition comprising a therapeutically effective amount of sorafenib

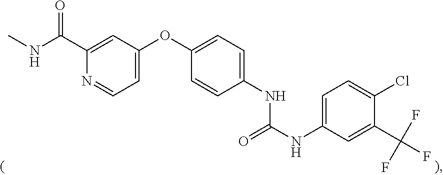 [SMILES: CNC(=O)c1cc(Oc2ccc(NC(=O)Nc3ccc(Cl)c(C(F)(F)F)c3)cc2)ccn1]

including pharmaceutically acceptable salts, solvates, and/or prodrugs thereof.

In certain embodiments, the present invention provides kits comprising a composition comprising 6-methoxyethylamino-numonafide (MEAN):

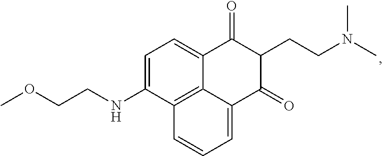

and instructions for administering such a composition to a patient having hepatocellular carcinoma.

In some embodiments, the composition is a pharmaceutical composition.

In some embodiments, the kits further comprise a composition (e.g.., a pharmaceutical composition) comprising sorafenib

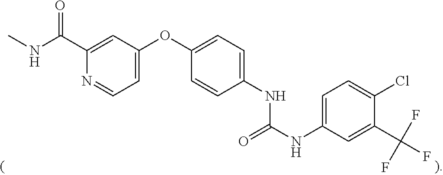 [SMILES: CNC(=O)c1cc(Oc2ccc(NC(=O)Nc3ccc(Cl)c(C(F)(F)F)c3)cc2)ccn1]

### BRIEF DESCRIPTION OF THE DRAWINGS

FIG. 1. MEAN inhibits Huh7-luc xenograft tumor growth more potently than sorefenib in nude mice. Ten days after inoculation of 10⁷ tumor cells subcutaneously, mice were treated with MEAN (15 mg/kg), sorafenib (15 mg/kg), or MEAN and sorafenib in combination at 15 mg/kg each, or vehicle through IP at a schedule of 5 days-on and 2 days-off for 42 days. The tumor growths were measured by whole mount imaging of luciferin florescence (representative images) (A) photon counts biweekly (B) and photon count at end of the experiment, day 42th (C), by volume (D), and by tumor weight at the experimental end point (E). Statistical analyses using t test are shown in (C and E), where *=p<0.5 and **=p<0.01. Where there is not black bar indicates p>0.5 (n=5).

FIG. 2. MEAN inhibits HepG2-luc xenograft tumor growth more potently than sorefenib in nude mice. Ten days after inoculation of 10⁷ tumor cells subcutaneously, mice were treated with MEAN (15 mg/kg), sorafenib (15 mg/kg), or MEAN and sorafenib in combination at 15 mg/kg each, or vehicle through IP at a schedule of 5 days-on and 2 days-off for 42 days. The tumor growths were measured by whole mount imaging of luciferin florescence (representative images) (A) photon counts biweekly (B) and photon count at end of the experiment, day 42th (C), by volume (D), and by tumor weight at the experimental end point (E). Statistical analyses using t test are shown in (C and E), where *=p<0.5 and **=p<0.01. Where there is not black bar indicates p>0.5 (n=5).

FIG. 3. Toxicity induced by treatment of MEAN, sorafenib or a combination of both were measured against vehicle treated group through monitoring body weight changes throughout treatment course (A) and detecting the serum levels of liver enzymes at the experimental end point (B). The recovery of liver enzymes days after experimental end point (42^{nd} day). n=10. a: each group vs Vehicle, b: sorafenib vs MEAN, c: M+S vs MEAN. *: p<0.05, **: p<0.01, Sorafenib vs M+S: has no difference.

FIG. 4. NCI 60 cell data analyses using COMPARE algorithm indicate distinct modes of action between MEAN and AMN. MEAN dose not correlate with any tested compounds in the database at the concentrations of LC₅₀ and GI₅₀ (A, top panel) (GI₅₀, 50%growth inhibition concentration, LC₅₀, 50% lethal concentration, TGI, total growth inhibition concentration). Comparison between MEAN and AMN does not show significantly correlation (A, bottom panel). Cell cycle analyses in HepG2 cells treated with MEAN, AMN, or DMSO demonstrate distinct cell cycle profiles between MEAN and AMN (B). However, MEAN is similar to AMN in the regard that MEAN is also not a substrate of activated p-glycoprotein, which is the common course of multi-drug resistance (C).

FIG. 5. MEAN and AMN differentially alter gene expression in treated HepG2 and Huh7 cells. RNA array analyses show that ⅔ gene expression changes in treated cells are not shared between MEAN and AMN (A). MEAN significantly reduces myc protein levels when using less than 10 uM while AMN does not significantly reduces myc expression at the same concentration (B and D). MEAN also reduces SIRT1 and increases SHP-1 protein levels while AMN does not significantly impact on the levels of these proteins. The intensity of vehicle is set at 1 and ratios of treatment/vehicle are plotted in each quantitative graph (B and D). Many downstream genes of myc reduce their expression levels correspondingly in MEAN or AMN treated cells except for two genes (C). The Y axis represents the log fold differences in RNA levels of each indicated gene between agents and DMSO.

FIG. 6. Sorafenib does not impact on the same gene expression as MEAN in vivo. Analyses using COMPARE algorithm with NCI 60 cell panel show that MEAN is unlikely to share mechanism of action with sorafenib (A). Western blot of tumor tissue treated by MEAN, Sorafenib, combination of both, or vehicle demonstrate MEAN suppresses proteins levels of myc and SIRT1 while increases protein levels of SHP-1 and TXNIP similarly to the findings in culture cells (B and C). Sorafenib does not impact on these gene expressions significantly (B and C). The intensity of vehicle is set at 1 and ratios of treatment/vehicle are plotted in each quantitative graph.

FIG. 7A-E. Pharmacokinetics (PK) of MEAN in mice after intravenous (IV, circles, continuous line), intraperitoneal (IP, squares, dashed line), and oral (PO, squares, dashed line) administration.

### DETAILED DESCRIPTION OF THE INVENTION

The primary curative therapy for hepatocellular carcinoma (HCC) is surgical resection with either a liver resection or liver transplantation¹⁰⁻¹¹. In Western countries, only 5% of patients with HCC are candidates for surgical resection and the only curative procedure for the remaining 95% of patients is liver transplantation; which is limited by the number of available donor livers and long waiting times. Adjuvant therapies for HCC such as percutaneous ablation, Transarterial Embolization and Chemoembolization (TACE) or Yttrium 90 microspheres¹² radiotherapy are often used as palliative therapies or as a “bridge” to liver transplantation. However, these treatments are typically non-curative. Systemic chemotherapy is not recommended before liver transplantation or as a “bridge” to transplantation due to the poor efficacy of available chemotherapeutic agents. The only approved chemotherapy for HCC is sorafenib, a tyrosine and serine-threonine kinase inhibitor that has been shown in two studies to have efficacy in treating HCC¹³⁻¹⁴. However, the effectiveness of sorafenib appears to be limited as median overall survival was only extended from 7.9 to 10.7 months in the SHARP study, and from 4.2 to 6.5 months in an Asian study. There was also significant drug toxicity in both studies¹³-15. In addition, sorafenib in combination with other chemotherapeutic agents have been disappointing¹⁶⁻¹⁷. The lack of effective systemic chemotherapy for HCC is the major reason for the poor 5 year survival rates for HCC patients in US (distant mediatized HCC 2%, regional HCC 7% and localized HCC 28%)².

Recently, 6-methoxyethylamino-numonafide (MEAN)¹⁸ , was synthesized and shown to be effective against HCC xenograft tumors in mice¹⁹. In the xenograft HCC models, MEAN was able to cause regression of established tumors over a 6 week treatment and animals tolerated the treatment very well, showing minimal toxicities¹⁹. MEAN is a derivative of Amonafide (AMN)

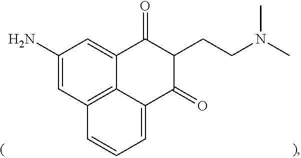 [SMILES: CN(C)CCC1C(=O)c2cccc3cc(N)cc(c23)C1=O]

an anti-cancer agent effective against a wide range of cancers that is not a substrate for multi-drug resistance drug efflux pumps. AMN has free aryl amine at the 5 position of the molecule that is acetylated by NAT2, forming N-acetyl AMN, which is toxic. The pharmacogenomics variability in the function NAT2 acetylation in the human population of AMN causes some patients to experience severe toxicities while others to be under treated at a set dose. The high and variable toxicity prevents AMN from FDA approval²⁰⁻²¹. MEAN

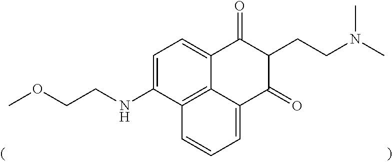

is an analogue of AMN with the aryl amine at the 6th-position blocked with an ethyl-methyoxy moiety.

While MEAN has similar efficacy as AMN in inhibiting growth of Huh7 and HepG2 HCC xenografts in mice, it has significantly less toxicity than AMN¹⁹. The differences in toxicity cannot be attributed solely to the inability of forming the toxic acetylated metabolites at the 5^{th} position since another derivative (Al) that also did not form the metabolite incurred similar toxicity as AMN, indicating that the reduction of toxicity of MEAN could be explained by distinct cellular mechanisms of action. As the only FDA approved systemic small molecule for HCC treatment is sorafenib, and to vet the future development of MEAN as a novel treatment for HCC, the anti-tumor efficacy and toxicities of the sorafenib and MEAN were compared in the experiments conducted during the course of preparing embodiments for the present invention.

Indeed, experiments conducted during the course of preparing embodiments for the invention determined that at the same concentration and treatment strategies, MEAN is more efficacious and less toxic than sorafenib. Treatment by MEAN at an efficacious dose was shown to not significantly impact animal body weight. Sorafenib in combination with MEAN was shown to inhibit tumor growth to a greater extent than single agent treatments and adding MEAN was shown to not significantly increase toxicities compared to sorafenib alone. Mechanistically, MEAN was shown to suppress c-myc expression and increase expression of several tumor suppressors, including SHP-1 and TXNIP. MEAN was shown to effectively inhibit cancer cell growth in several drug resistant cell lines with activated P-glycoprotein drug efflux pumps and was shown to have a drug like single dose pharmacokinetic profile. Altogether, these experiments demonstrate that MEAN is effective against HCC tumor growth as monotherapy and in combination with sorafenib, and is an excellent candidate for clinical development as a therapeutic agent for HCC management.

Accordingly, provided herein are compositions and methods for treating, ameliorating, or preventing hepatocellular carcinoma in patients. In particular, the invention relates to methods of treating, ameliorating, or preventing hepatocellular carcinoma in a patient, comprising administering to the patient 6-methoxyethylamino-numonafide alone or in combination with sorafenib.

In certain embodiments, the present invention provides compositions comprising 6-methoxyethylamino-numonafide (MEAN):

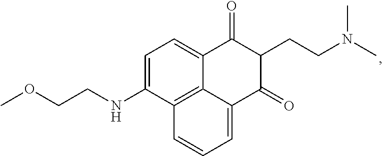

and sorafenib

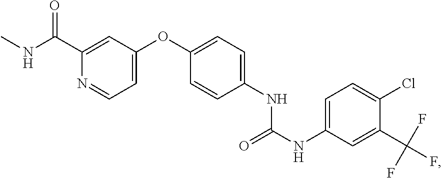 [SMILES: CNC(=O)c1cc(Oc2ccc(NC(=O)Nc3ccc(Cl)c(C(F)(F)F)c3)cc2)ccn1]

including pharmaceutically acceptable salts, solvates, and/or prodrugs thereof.

In certain embodiments, methods are provided for treating, ameliorating, or preventing hepatocellular carcinoma in patients through administration of therapeutically effective amount of MEAN (e.g., a composition comprising MEAN) (e.g., a pharmaceutical composition comprising MEAN) to the patient. In some embodiments, the methods further comprise co-administration of a therapeutically effective amount of sorafenib (e.g., a pharmaceutical composition comprising sorafenib) to the patient.

An important aspect of the present invention is that MEAN was shown to be able to reduce c-myc expression and increase SHP-1 and TXNIP expression (see, Examples). As such, the present invention provides methods wherein administration of a composition comprising MEAN results in a reduction of c-myc expression and an increase of SHP-1 and TXNIP expression.

In some embodiments, the methods for treating, ameliorating, or preventing hepatocellular carcinoma in patients through administration of therapeutically effective amount of MEAN involve c-myc expression and an increase of SHP-1 and TXNIP expression in the patient (see, Examples).

In some embodiments, additional anti-cancer agents are co-administered to the patient (e.g., any type or kind of chemotherapy and/or drug therapy and/or radiation therapy).

One of ordinary skill in the art will readily recognize that the foregoing represents merely a detailed description of certain preferred embodiments of the present invention. Various modifications and alterations of the compositions and methods described above can readily be achieved using expertise available in the art and are within the scope of the invention.

### EXAMPLES

The following examples are illustrative, but not limiting, of the compounds, compositions, and methods of the present invention. Other suitable modifications and adaptations of the variety of conditions and parameters normally encountered in clinical therapy and which are obvious to those skilled in the art are within the spirit and scope of the invention.

### Material and Methods:

Human HCC cell lines HepG2 and Huh7 were maintained in Dulbecco's Modified Eagle Medium (DMEM, Gibco) containing 10% fetal bovine serum (FBS, Gibco), 100 Units/mL Penicillin, and 100 ug/mL Streptomycin (Thermo Fisher Scientific). HepG2-luc and Huh7-luc cell lines were constructed as our previous study with little modified¹⁹, and were also cultured in DMEM supplemented with 10% FBS.

### Mouse Modeling:

HepG2-luc and Huh7-luc xenograft studies of efficacy and toxicity: Male nu/nu (nude) mice (18-20 g at experiment initiation) were maintained at the vivarium of Zhejiang University in a pathogen-free unit, under a 12-hour light/dark cycle. Mice were inoculated subcutaneously in the right axilla with HepG2-luc or in the left axilla with Huh7-luc (10⁶ cells). After 10 days of tumor growth, mice were randomized into groups of 5 mice prior to drug treatment. Sorafenib (LC Laboratories) was dissolved in 1:1 ethanol and cremophor EL (Sigma-Aldrich) and then diluted in PBS to 5 mg/ml on the day of treatment. MEAN was also dissolved in 1:1 ethanol and cremophor EL (Sigma-Aldrich) and then diluted in PBS to 2 mg/ml, and stored at −20° C. Sorafenib (15 mg/kg) or MEAN (15 mg/kg) was administered by intraperitoneal injection for five consecutive days followed by two days without dosing. Vehicle control was 10% 1:1 ethanol and cremophor EL (Sigma-Aldrich). In vivo bioluminescent imaging to determine tumor burden was performed with a Lumina imaging system (Nippon Roper, I.C.E., Tokyo, Japan). Ten minutes before imaging, mice were injected with 150-mg/kg luciferin through an intraperitoneal route. Images were collected and analyzed with Living Image software (Slidebook 4.1, Denver, Colo.). Tumor volumes were determined twice weekly by mechanically measuring the length (a) and width (b), with volume=ab²/2. Tumor weights were measured at the experimental end point, and statistical analysis was performed using Student's t test.

### ALT and AST Measurement:

Blood samples were collected from each mouse at the end of the 42th day. Serum alanine aminotransferase aspartate (ALT) and aspartate aminotransferase (AST) were measured with an automated biochemical analyzer (DRI-CHEM 4000ie, FUJIFILM).

### RNA Array Analyses in HepG2 Cells:

RNA was isolated from 10⁷ HepG2 cells with Trizol 6 hrs after treatment with 20 μM of AMN, MEAN, or vehicle (0.2% DMSO). RNA expression analysis was performed by GeneChip® PrimeView™ Human Gene Expression Array. Initially, genes were identified as being differentially expressed on the basis of a statistical significance (P <0.05) and 1.5-fold change (up or down) in expression levels in each comparison.

Cell Cycle Profiling using Dapi Staining

After treatment, cells were washed with PBS two times and centrifugation then, re-suspended in PBS (Ca and Mg free). 3.0m1 ice cold 95% ethanol was added to the cell pellet in a dropwise manner while vortexing and then was incubated for 30 minutes. The volume was brought up to 15 mL with PBS and the cells was pelleted and washed one time with PBS via centrifugation. Cells were resuspended in 1 ml of 10 ug/ml Dapi in PBS with 0.1% TritonX-100 to a concentration of 1×10⁶cells/ml and incubated for 30 minutes on ice. The samples were then analyzed through flowcytometery and the DNA contents of the cellular population were sorted and evaluated.

### Western Blotting:

Cells or tumor chunks were lysed with RIPA lysis buffer (Beyotime Biotechnology, Jiangsu, China) containing 1% Halt™ Protease and Phosphatase Inhibitor Cocktail (100X) (Thermo Fisher Scientific). 20 ug of total protein per lanewere separated by 10% SDS-PAGE gels and transferred to polyvinylidene difluoride membranes (Millipore, Bedford, Mass.), and blocked with 1% BSA in TBS-Tween 20 (0.05%, v/v) for 1 h at room temperature. The membrane was incubated with primary antibody overnight at 4° C. Antibodies for c-Myc and GAPDH were obtained from Cell Signaling Technology (Beverly, Mass.), and TXNIP, SHP-1, and SIRT1 were obtained from from Abcam (Cambridge, Mass.). After washing, the membrane was incubated with the appropriate horseradish peroxidase-conjugated secondary antibody (1:3000; Eptmomics) for 1 h. Blots were visualized by ECL-associated fluorography (Millipore). Relative band intensity was quantified using the ImageJ software to determine c-myc, TXNIP, SHP-1, and SIRT1 levels.

### Statistical Analysis

Statistical analysis was performed with T-test to evaluate differences between groups. Each in vitro experiment was repeated at least three times and the data were represented as mean±SE. p<0.05 was considered significant.

### Results:

### MEAN is More Efficacious Than Sorafenib at the Same Concentration in HCC Xenografts.

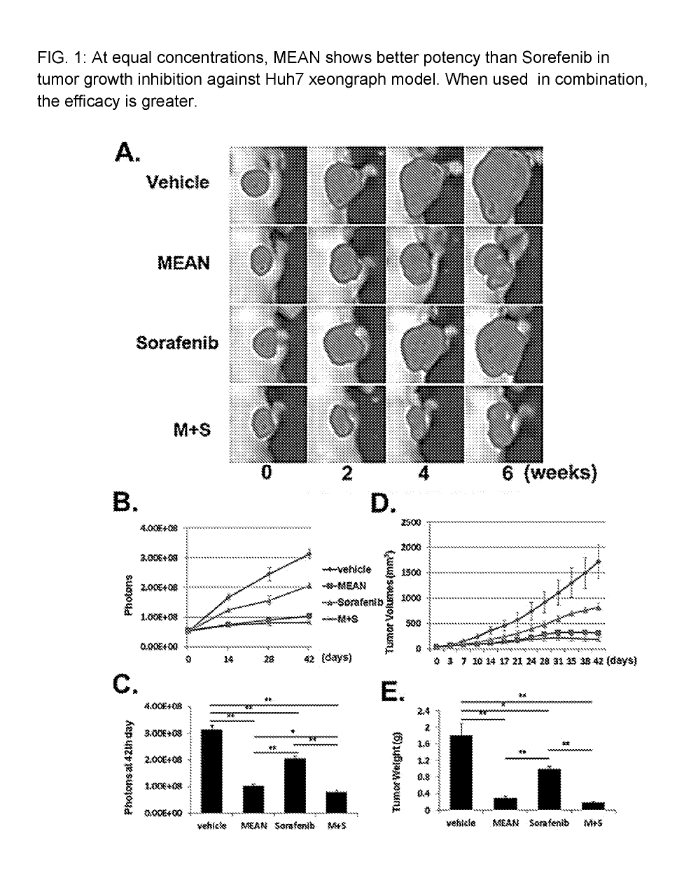

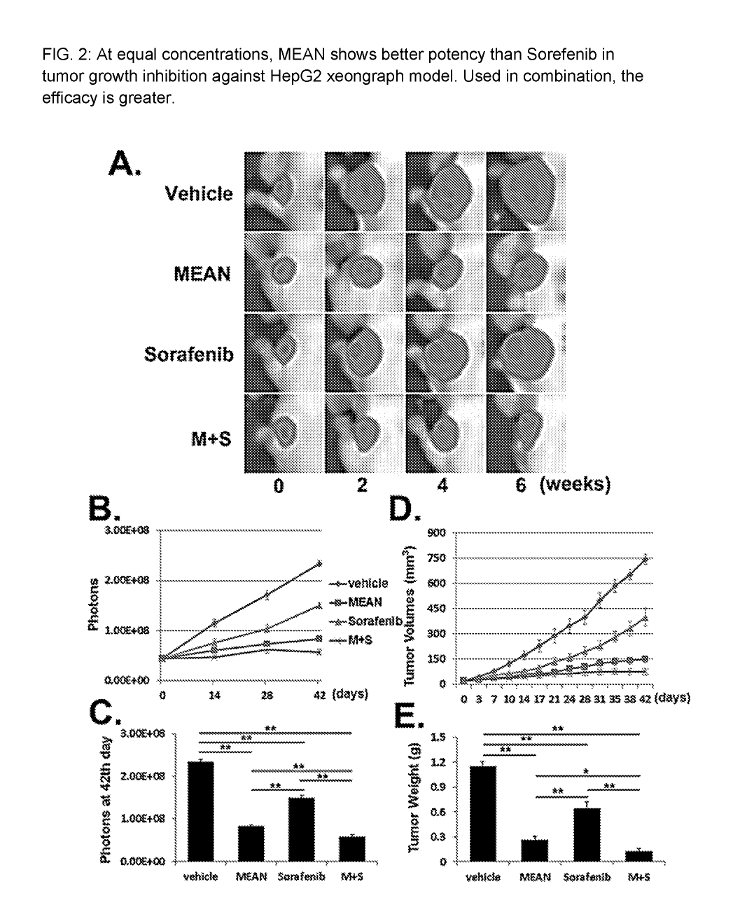

We have previously shown that MEAN, at 50 or 100 umol/kg (17.1 mg/kg or 34.2 mg/kg respectively), was effective in against Huh7 and HepG2 xenografts in mice¹⁹. To directly compare the efficacy of MEAN with the only FDA approved anti-HCC agent, sorafenib, both agents were used to treat the same HCC xenograft models at the same treatment strategies. Nude mice were inoculated subcutaneously with 10⁶ of Huh7-luciferase or HepG2-luciferase cells, and tumors were allowed to grow for 10 days. Drug treatment was initiated on day 11^{th} with either MEAN or sorafenib at 15 mg/kg with a schedule of 5 days-on and 2 days-off for 42 days. Evaluation of the luciferase emission biweekly (FIG. 1A and 1B, 2A and 2B), tumor volume biweekly (FIG. 1D and 2D), or tumor weight at the experimental end point (FIG. 1E and 2E) demonstrate that treatment with both agents at given concentrations show significant tumor growth retardation in treated mice compared with control mice injected with vehicle only (FIG. 1 and FIG. 2) (p<0.05 for both agents in tumor growth compared to vehicle treated animals). Although the established tumor did not shrink as shown previously with higher concentrations of MEAN¹⁹, the 15 mg/kg MEAN treatment nearly entirely block the tumor growth (FIG. 1). With this treatment regime, MEAN is significantly more effective than sorafenib in tumor growth inhibition (p<0.05) for both HCC xenograft tumor models in all the manner of measurements (FIGS. 1 and 2).

### MEAN and Sorafenib Combination Enhances Efficacy in HCC Xenograft .

As both MEAN and sorafenib show significant efficacy against tumor growth in two human HCC xenograph tumor models, the question became whether the combinational treatment with both compounds could be more effective. The same concentration of both agent MEAN and sorafenib (15 mg/kg) were used in combination to treat Huh 7 and HepG2 xenograft tumors in parallel with single agent treatment. The results show that the combination of MEAN and sorafenib demonstrates a trend towards greater efficacy in both tumor models. The combination flattens tumor growth and begins to reduce tumor size in both models around day 30^{th} after the treatment began (FIGS. 1 and 2). While the combination is significantly more efficacious than sorafenib treatment alone, it is much less significantly when compared to MEAN monotherapy (FIGS. 1 and 2). The modest enhancement of MEAN efficacy by addition of sorafenib could be due to that MEAN efficacy is reaching near maximal inhibition at 15 mg/kg and adding a low concentration of sorafenib may not matter as much.

Combination of MEAN with Sorafenib Does Not Significantly Increase Toxicity

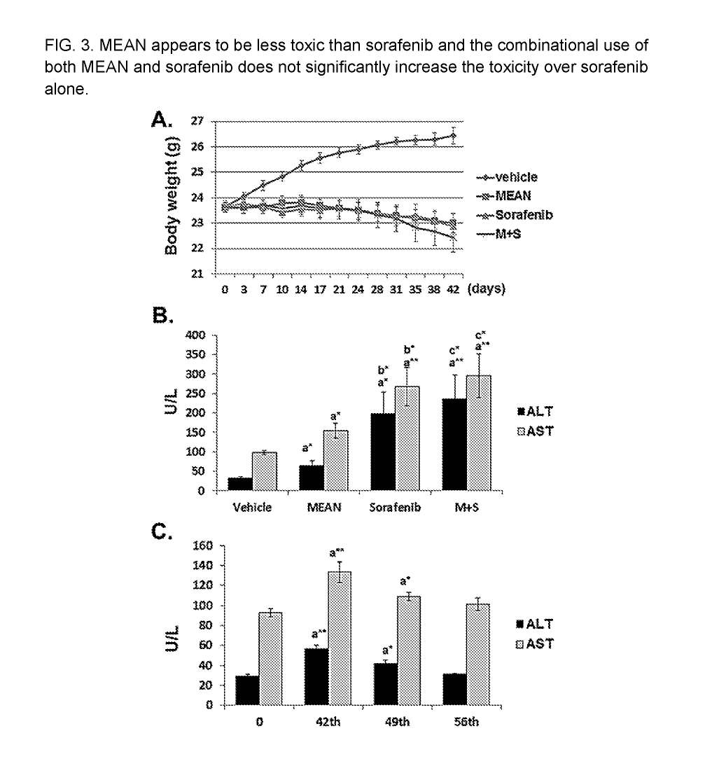

To determine whether the combinational treatment by MEAN and sorefenib could increase toxicity compared to those caused by monotherapy, the toxicity induced by the treatment with either agent alone and in combination was evaluated in treated animals. Toxicity was assessed by periodic body weight, mice behavior (grooming), eating, stool consistency, and serum liver enzymes levels (alanine aminotransferase, ALT, and aspartate aminotransferase, AST) at the experimental end points. Through the six weeks treatment with a schedule of 5 days on and 2 days off by IP injections, mice treated with MEAN at 15 mg/kg did not show significant changes in body weight (<3%) from those of prior to treatment (FIG. 3A) while vehicle treated mice show escalated body weight primarily from the tumor growth. Liver enzyme quantification at the experimental endpoint shows that a significant increases after 42 days of treatment (p<0.05) (FIG. 3B). This increase is reversible within 2 weeks of the treatment withdrawal (FIG. 3C). Mice in the treatment and vehicle groups remained equally active, and had normal stool consistency, food intake and grooming. These results indicate that at the effective tumor inhibitory treatment concentration, MEAN does not induce obvious adversary effect onto treated animals. In comparison, parallel treatment with sorafenib at 15 mg/kg with the same schedule also did not significantly alter body weight or living quality of treated animals. However, ALT and AST were more significantly increased comparing to vehicle treated animals at the 42^{nd} day of the treatment (FIG. 3B, p<0.01) and were significantly higher than MEAN monotherapy. These data demonstrate that while less effective in tumor growth inhibition, sorafenib is more toxic than MEAN. When mice were treated with the combination of MEAN (15 mg/kg) and sorafenib (15 mg/kg) with the same 5 days on and 2 days off schedule, their body weight did not significantly change (FIG. 3A) with the exception of one mouse that showed more pronounced body weight loss and less activity. However, the liver enzymes were significantly higher than vehicle or MEAN monotherapy treated mice, but were not significantly higher than sorafenib treated mice (p>0.05). Similarly, the combination of MEAN and sorafenib did not significantly add to the toxicity of sorafenib monotherapy while blocking tumor growth almost completely (FIGS. 1 and 2).

### MEAN Shows Distinct Mode of Action Compared to the Parental Compound AMN.

As MEAN a derivative of AMN, the two compounds share tumor growth inhibition efficacy and some of the molecular mechanisms of action (Topo II inhibition). However, differences in activity observed in vivo in previous studies suggest that MEAN may possess additional unique molecular mechanisms of action¹⁸⁻¹⁹. There is an increasing awareness of multi-pharmacology, in which many small molecules often have more than one target, including those that have been developed specifically against a single target. These unintended effects have been historically termed off-target effects. However there is little evidence whether the off-target effects could contribute to the desirable outcomes from the treatment by these molecules.

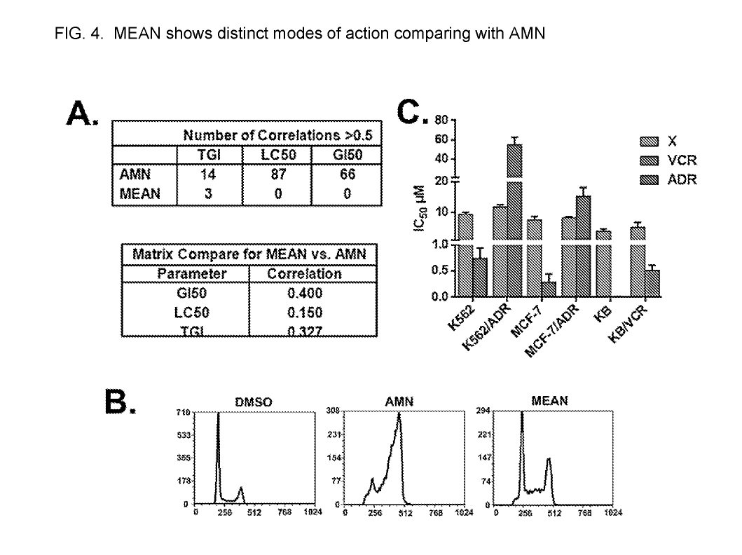

To evaluate similarity and differences of the mode of action between MEAN and AMN, the COMPARE algorithm was used to analyze the data resulting from the tests of these compounds in the NCI 60 cell line panel growth inhibition assays. The COMPARE algorithm²² gives a correlation value close to 1 when two drugs have a similar pattern of inhibition in the 60 cell line panel, and when there is no correlation, a value of zero, and −1 when there is an inverse correlation (range −1 to 1). The COMPARE algorithm for AMN verses the NCI's Standard, showed that there are 14, 87, and 66 drugs with correlations greater than 0.5 based on total growth inhibition concentrations (TGI), 50% lethal concentrations (LC₅₀), and 50% growth inhibitory concentrations (GI₅₀). In contrast, there were no correlations greater than 0.5 for MEAN based on LC₅₀ and GI₅₀ and only 3 correlations based on TGI (FIG. 4A, top panel) (Supplemental material Table 1). The majority of compounds with high correlations with AMN are DNA damaging or DNA binding agents, indicating genome toxicity to be the main anticancer mechanism for AMN, which is consistent with the literature²³. In contrast, the low correlation values and small number of correlative compounds for MEAN with values greater than 0.5 (FIG. 4A) suggest that genome toxicity is not the main mode of action for MEAN, differing significantly from AMN. Furthermore, when MEAN is compared to AMN directly, the correlation scores are <0.5 at all three (LC₅₀, GI₅₀, and TGI) concentrations (FIG. 4A bottom panel), indicating more difference than similarity in the tumor growth inhibition by MEAN comparing to AMN.

Additional evidence supporting the differences between MEAN and AMN came from their differential impact on cell cycle. HepG2 cells were treated with MEAN and AMN at the same concentration of 5 uM for 24 hours and the cell cycle profiles were evaluated by DNA contents through flow cytometry. The results demonstrate significant differences (FIG. 4B). While AMN induces G2 accumulation with diminishing Gl, MEAN treatment shows significant retention in S phase, but not as much in G2. These findings are supportive that MEAN possesses novel anti-tumor growth mechanisms, which may contribute to its tumor growth inhibition with significant lower toxicity in vivo, comparing to AMN.

However, MEAN is similar to AMN such that MEAN is also not a substrate of activated P glycoprotein, which is a common cause of drug resistance. To address this point, the IC50 of MEAN in cell lines with or without activated p-glycoprotein were compared based on the rationale that if an agent is a substrate, its IC₅₀ (growth inhibition in this case) will increase substantially in cells with activated p-glycoproteins (multi-drug resistant cells). Three separate experiments were carried out to determine the values of IC₅₀, as mean±standard deviation (μM), and the inhibition rates of each concentration of compounds were tested in triplicate. The resistance factor (RF) was calculated as the ratio of the IC₅₀ value of the multidrug-resistant cells to that of the corresponding sensitive parental cells.

IC₅₀ of MEAN and reference compounds, doxorubicin and vincristine (VCR), were evaluated in three MDR expressing sublines: K562/ADR, MCF-7/ADR, and KB/VCR developed from resistance to Adriamycin (ADR) (FIG. 4C). For each of these cell lines, the RFs were measured to indicate the anti-MDR properties of the compounds. The resistance factor for doxorubicin in K562/ADR and MCF-7/ADR sublines and for vincristine in the KB/VCR subline were 74.1, 54.0, and 51.2. In comparison, MEAN displayed equal potent growth inhibition between the multidrug resistant cells and their corresponding drug sensitive parental cells, with RF values of 1.1-1.3. These data demonstrate that the growth inhibition efficacy of MEAN is not reduced in multi-drug resistant cell line, indicating apparent anti-MDR activity of MEAN.

### MEAN Significantly Changes Gene Expression Profiles Both in Vitro and in Vivo

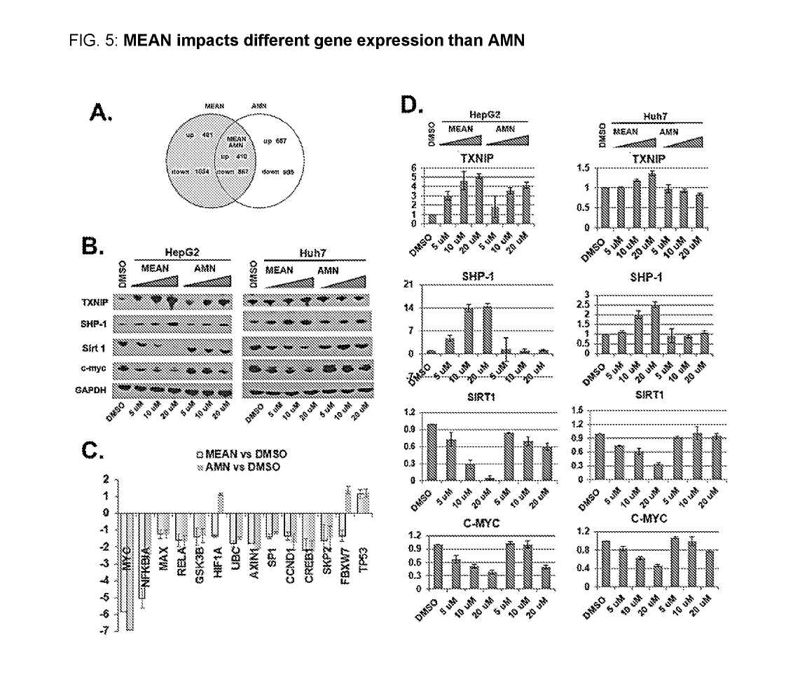

To determine the uniqueness of the mode of action, through which MEAN inhibits HCC tumor growth, genome expression profiles at the RNA level in HepG2 cells treated with MEAN, AMN or DMSO were examined and compared. HepG2 cells were treated by MEAN or AMN at the concentration of 20 μM for 6 hours or DMSO treated cells. The choice of the treatment dose was based on the PK analyses (supplemental material) that the serum concentration of MEAN is around 20 uM approximately 6 hours after injection. Furthermore a relatively short duration of treatment was designed to catch the more primary cellular responses at the early stage of the treatment. GeneChip® PrimeView™ Human Gene Expression Array was used in triplicates to quantify RNA extracted from treated HepG2 cells. The results of these experiments demonstrate that while MEAN and AMN share about ⅓ of impacted genes, they also regulate distinct sets of genes (FIG. 5A, the number of genes that change more than +/−1.5 log fold).

Based on results from the array analyses, we experimentally confirmed some of the expression changes in cell lines. While MEAN and AMN both significantly reduce c-myc RNA expression level in treated cells, MEAN shows more significant suppression of c-myc protein expression in treated cells than AMN (FIG. 5B and D). As c-myc is well-known for its oncogenic role in HCC specifically²⁴ and in cancers generally²⁵⁻²⁷, reduction of c-myc in treated cancer could significantly contribute to tumor growth suppression. To determine the downstream effect of c-myc in treated cells, the RNA levels of some of the downstream targets were evaluated. As demonstrated in (FIG. 5C), while majority of downstream elements of c-myc are shown to be similarly regulated by MEAN and AMN, two genes (HIF1A and FBXW7) are exceptions, suggesting that some of these factors might also be regulated by other mechanisms.

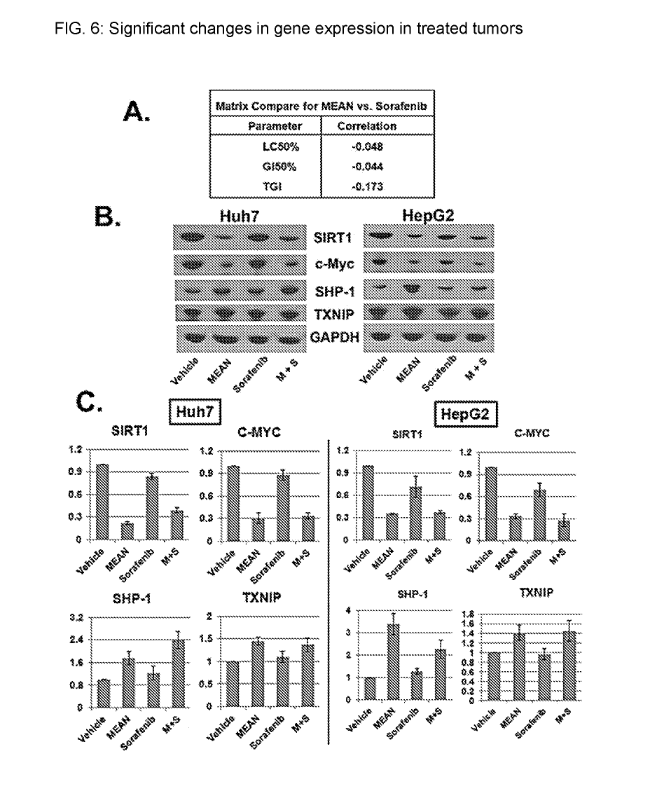

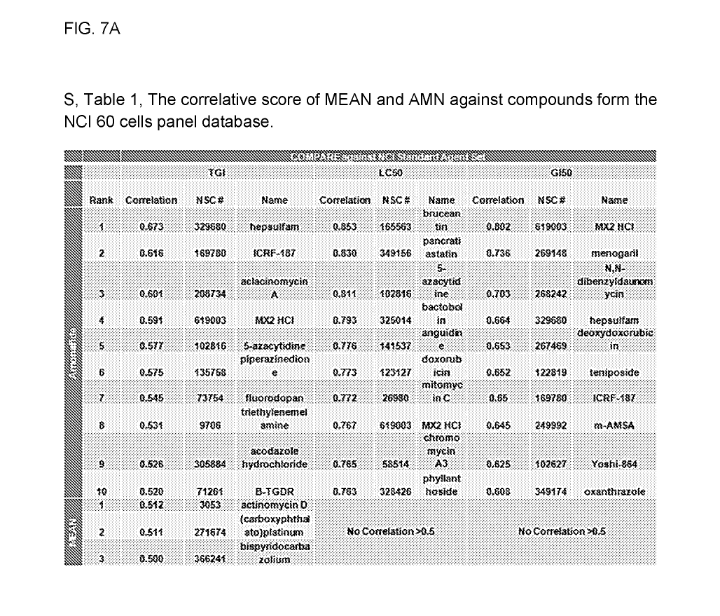

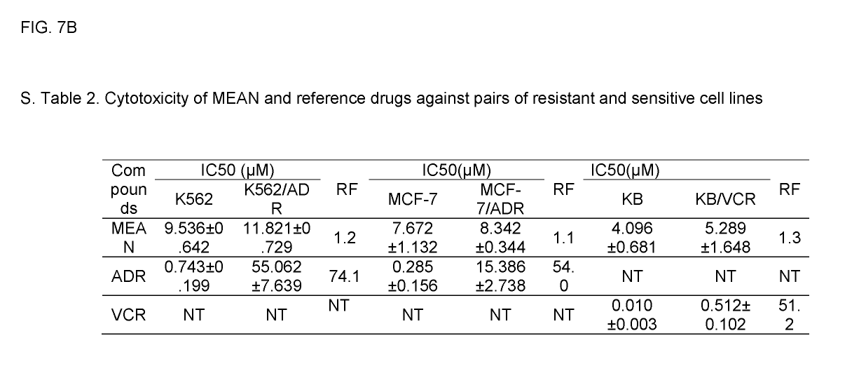

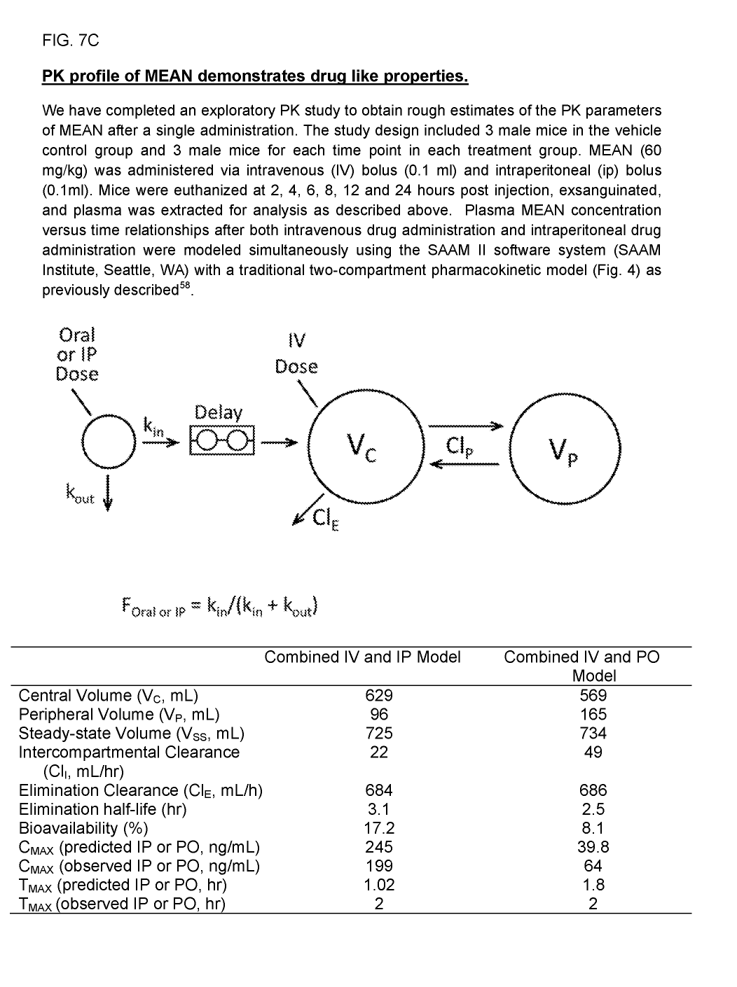

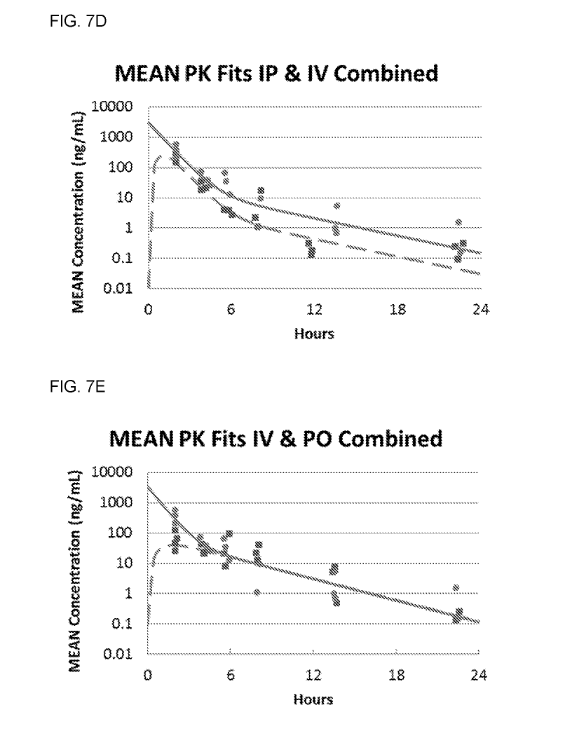

To evaluate whether the gene expression changes in response to MEAN is cell type specific, another HCC cell line, Huh7 was also examined for the corresponding gene expression changes upon the treatment by MEAN. The results show similar changes of the protein levels of c-myc and other factors in these cells (FIG. 5B and D). To determine whether the similar gene expression is also true in tumors that were treated with MEAN, the resected tumor tissues from animals were examined for protein levels of the corresponding genes and the results from tissues that are treated with either MEAN or vehicle were compared. The results show that c-myc is significantly reduced in both Huh7 and HepG2 tumors at the end of 42 day treatment course by MEAN, but not in those treated by vehicle (FIG. 6B and C). These findings demonstrate a consistent reduction of c-myc levels in response to MEAN treatment both in vitro and in vivo.

In addition to c-myc, a few other gene expressions are also reduced. SIRT1 has been shown to be highly elevated in HCC tumors²⁸⁻²⁹. Western blot analyses of HepG2 and Huh7 cells show that the levels of SIRT1 protein are significantly reduced in MEAN treated cells, but are not significantly changed in AMN treated cells (FIG. 5B and D). Evaluation of treated tumors in vivo confirmed the reduction of SIRT1 in treated tumors (FIG. 6B and C). These findings indicate that SIRT1 is specifically reduced in MEAN treated HCC cells and tumors, differing from AMN.

Is it possible that the reduction of c-myc reflects general transcription shutdown in treated cells? Even through MEAN does not significantly correlate with genome toxic compounds except for a few, including actinomycin D, at the total growth inhibition concentration, it is important to distinguish whether c-myc reduction results from a general transcription decrease or from specific regulators targeted by MEAN. To do so, gene expressions across the genome were evaluated through RNA array analyses and the results show that many factors and RNAs are upregulated in response to MEAN treatment as compared to the controls (FIG. 5A), supporting the idea that the reduction of a subset of gene expression is not due to a global inhibition of transcription.

Several proteins are upregulated in response to MEAN treatment. One of the proteins that are upregulated is SHP-1, a protein-tyrosine phosphatase, which has been shown to play roles in tumor suppression of HCC³⁰⁻³¹. Treatment by MEAN, but not by AMN or sorafenib induces significantly increases in the protein levels of SHP-1 in HepG2 and Huh7 cells in culture (FIG. 5B and D), or in treated tumors (FIG. 6B and C). Another tumor suppressor TXNIP is also selectively and significantly enhanced in MEAN treated cells both in vitro and in vivo (FIGS. 5 and 6).

These findings together demonstrate that MEAN has distinct mechanisms of action from its parental compound AMN. The expression of genes impacted by MEAN treatment are not all in the same cellular pathways or functions, indicating the likelihood of multi-targets and multi-modes of action for MEAN in tumor growth inhibition.

### MEAN and Sorafenib Do Not Appear to Share Mechanisms of Action in Tumor Growth Inhibition.

Combinational treatment using MEAN and sorafenib shows enhanced efficacy and addition of MEAN does not significantly increase liver toxicity of sorafenib, making the combination treatment an alternative strategy to enhance efficacy and reduce toxicity with further optimization of dosing. To distinguish withether the two compounds are additive or synergistic, one must first address whether the two compounds share mechanisms of action. Sorafenib is a well-interrogated FDA approved drug and is derived from screens against a protein tyrosine kinase³²⁻³³. However, sorafenib could also have other mechanisms of action in cells. The COMPARE algorithm²² was used to analyze the NCI-60 cell line data for MEAN and sorafenib. A direct comparison between MEAN and sorafenib yields correlation scores of negative numbers at all three concentrations, LC₅₀, GI₅₀ and TGI (FIG. 6A), suggesting the unlikeliness that MEAN and sorafenib share common mechanisms of action in tumor growth inhibition. To further assess this idea, we evaluated the proteins levels that were shown to be affected by MEAN in sorafenib treated tumors. As shown in FIG. 6, sorafenib treatment does not significantly change the levels of proteins, including c-myc, SIRT1, SHP-1, or TXNIP, all of which are impacted in MEAN treated tumors (FIG. 6B and C). Furthermore, when HCC xenograft mice models were treated by the combination of MEAN and sorafenib, the levels of these factors do not show significant differences from those treated by MEAN alone. The combinational treatment also does not incur significantly more toxicity to animals than sorafenib monotherapy. These observations together support that MEAN and sorafenib do not appear to share common mechanisms of action in tumor inhibition.

### Discussion:

Human HCC is a major health problem in the US and the world. The lack of effective chemotherapeutic intervention significantly contributes to the high mortality from the disease particularly as donor livers for curative liver transplants are limited by the supply. Sorafenib, the only FDA approved small molecule drug, shows very limited efficacy with substantial toxicity, which render development of novel therapeutic agents urgent and necessary. Here we report that a small molecule, MEAN, is effective in tumor growth inhibition against two HCC xenograft tumor models without significant toxicity to treated animals. At the same concentration, MEAN shows more potent efficacy in tumor growth inhibition than sorafenib in both HepG2 and Huh7 xenograft models, and induces less toxicity than sorafenib. When used in combination, the tumor growth inhibition is greater than sorafenib or MEAN used alone without significantly increases in toxicity over sorafenib monotherapy, providing a potentially enhanced therapeutic strategy for HCC patients. Mechanistically, MEAN significantly reduces c-myc oncogene expression in treated tumors and impacts other gene expression levels that could also contribute to its tumor inhibition mechanism. It does not appear to share common mode of action with sorafenib. Furthermore, MEAN is not the substrate of activated p-glycoprotein, a common cause for drug resistance. With drug like pharmacokinetics, MEAN has a great potential to be further developed into a first line and/or second line drug for the treatment of human HCC.

MEAN Inhibits Tumor Growth with Distinct Modes of Action From AMN.

MEAN is a derivative of a well interrogated anti-cancer compound AMN. AMN has been shown an anti-cancer agent effective against a wide range of cancers without the risk of multi-drug resistance. In spite of a large number of clinical trials, the severe and variable toxicity of AMN has prevented its further development^{20-21,23,34}. It is believed that the acetylated metabolite of AMN at the 5^{th} position by liver N-acetyltransferase 2 (NAT2) is responsible for the toxicity. MEAN is an analogue of AMN with the nitrogen moved from the 5-position to the 6-position¹⁸, preventing it from being acetylated by NAT2. While MEAN has similar efficacy as AMN in tumor growth inhibition against Huh7 and HepG2 HCC xenograft tumors in mice, it has significantly less toxicity in vivo¹⁹.

Our results here demonstrate unique differences in the mechanisms of action between MEAN and AMN. 1) The NCI 60 cell line panels COMPARE analyses do not show correlations (r<0.5) between MEAN and AMN or other known genome toxic compounds at the GI₅₀ or IC₅₀ concentrations (FIG. 4A). 2) There are significant differences in the genome expression profiles of HepG2 cells when treated with MEAN vs. AMN (FIG. 4B). 3) Furthermore, when protein levels from culture cells and tumors treated with MEAN were compared with those treated with AMN or vehicle, differences in a subset of protein expression levels were observed in cells after the corresponding treatment (FIG. 5). These findings demonstrate that although MEAN is a structural analogue of AMN, it has different mechanisms of function that could contribute to its anti-tumor effects and lower toxicities in vivo. These findings also indicate that MEAN possesses novel anti-tumor activities.

### Tumor Growth Inhibition Mechanisms of MEAN

Although direct targets of MEAN remain unclear, MEAN has been shown to disrupt topo II activities similarly to AMN¹⁸. Our genome profiling studies using HepG2 cells demonstrate that approximately ⅔ of genes that change more than 1.5 fold are regulated differently by MEAN and AMN. Interestingly, both MEAN and AMN significantly reduces c-myc expression although more significantly by MEAN than by AMN (FIG. 5). The notorious oncoprotein has been shown a key player in oncogenesis²⁵⁻²⁷ and the over expression of c-myc has been linked to human HCC^{24,35,36}. From these known function of c-myc in HCC, it is highly possible that the impact on c-myc could significantly contribute to the tumor growth inhibitory function for both compounds. Both MEAN and AMN also significantly increase the expression of thioredoxin-interacting protein (TXNIP). TXNIP is a multifunction tumor supressor³⁷⁻⁴⁰, implicated in ROS metabolism⁴¹⁻⁴³, glucose homeostasis⁴⁴, p53 stabilization⁴⁵⁻⁴⁶, and tumor suppression through inhibition of mTORC1⁴⁷. Induction of TXNIP has been shown to suppress the growth of HCC tumors⁴⁸. A recent report of an anti-tumor small molecule also has the effect of increasing TXNIP⁴⁹. While TXNIP functions opposite of c-myc⁴⁰, no studies so far demonstrate any co-regulation of or direct interactions between the two, suggesting that they could be regulated through different pathways by MEAN in HCC cells.

Unique to MEAN, we found that the treatment enhances the expression of SHP-1, a protein tyrosine phosphatase that has been shown to be both a tumor suppressor and an enhancer 31, 50-51. SHP-1 is a multifunctional protein, involved in glucose metabolism, autophage activation etc.^{30,32-53}. A recent report shows that the activation of SHP-1 significantly represses HCC colony formation in vitro and inhibits xenograft HCC tumor growth in vivo³⁰. Thus the significant activation of SHP-1 by MEAN could contribute to the tumor growth inhibition. Furthermore, MEAN inhibits SIRT1 expression. SIRT1 is a member of sirtuins deacetylases family with substrates from histone to enzymes involved in glucose metabolism⁵⁴. SIRT1 has diverse roles in hemostasis of many cellular processes, including energy metabolism, oxidation, Wnt, TGF-b, and NF-kB signaling pathways, and tumor suppressing⁵⁴⁻⁵⁵. SIRT1 is often found overexpressed in various cancer cells⁵⁶. It is thought that SIRT1 may regulate TERT and promote c-myc activities in HCC cells⁵⁷. SIRT1 is shown to be overexpressed and knockdown of SIRT1 induces HCC cell growth arrest²⁸. Therefore, the reduction of SIRT1 expression mediated by MEAN could help reduce c-myc expression and contribute to tumor growth suppression.

Our findings so far have identified gene expression changes in favor of tumor growth suppression. Looking into the specific up- or down regulation of genes, not all of them have direct known association with each other. Several of these proteins are regulated by more than one pathway and have multifaceted functions in cells. These findings support that MEAN is likely to inhibit tumor growth through impacting multiple cellular targets and the sum of these effect could be responsible for the tumor growth inhibition in vitro and in vivo. The idea is consistent with multipharmocology of a single compound, where an agent effects multiple cellular processes and the concerted outcomes yield the desirable treatment results. Future studies will investigate the key mechanisms that are the main contribution to MEAN mediated tumor growth inhibition.

### MEAN is More Potent in Tumor Growth Inhibition and Incurs Less Toxicity Than Sorafenib, and the Combinational Use Enhances Efficacy.

As sorafenib is the only drug approved by FDA for HCC treatment, it was used as a standard to compare MEAN's potency and toxicity in treating HCC xenograft tumors in mice. In these studies, animals bearing HepG2 and Huh7 xenograft tumors were treated by MEAN or sorafenib in parallel at the same concentrations with the same schedules. The results demonstrate significant stronger growth inhibition by MEAN over sorafenib, indicating MEAN as monotherapuetic agent is more effective than sorafenib in mice (FIGS. 1 and 2). Furthermore, sorafenib incurs more toxicity as evaluated in the significantly elevated liver enzymes at the experimental endpoint. These findings demonstrate that MEAN has potentials for being further developed into an effective monotherapeutic agent against HCC. When used in combination, the tumor growth inhibition is greater than either one used alone, although much more significantly when compared with sorafenib rather than with MEAN (FIGS. 1 and 2).

MEAN and sorafenib do not appear to act through the same mechanisms of action for tumor growth inhibition. Our observations demonstrate that there is no correlation between MEAN and sorafenib using the COMPARE algorithm at all treatment concentrations, suggesting that they inhibit tumor growth through different paths. Furthermore, evaluations of the impact by sorafenib on the protein levels shown to be changed by MEAN demonstrate mostly non-overlapping effects by the two agents on these proteins. The differences in mechanisms of action are beneficial for the combinational use of the two compounds which could help enhance the efficacy and reducing of toxicity.

In summary, we have demonstrated a novel small molecule that is effective at inhibiting tumor growth in HCC xenograft models. Monotherapy using MEAN at the same treatment strategy and concentration shows a more superior tumor growth inhibition efficacy with less toxicity, comparing to the sole FDA approved drug sorafenib. A combinational treatment with the agents yields greater tumor growth inhibition without significant increases in toxicity. MEAN treatment significant reduces well-known oncoprotein c-myc and other proteins know to act as tumor promoters such as SIRT1. At the same time, it increases proteins that are known to suppress tumor growth, including TXNIP and SHP-1. MEAN has distinct mode of action from AMN and does not share mechanisms of action with sorafenib. Furthermore MEAN is not the substrate of p-glycoprotein, a common cause for drug resistance. With a drug like PK profile, MEAN is a good candidate to be further developed into monotherapeutic or combinational therapeutic with sorafenib for human HCC treatment.

### INCORPORATION BY REFERENCE

The entire disclosure of each of the patent documents and scientific articles referred to herein is incorporated by reference for all purposes.

### Equivalents

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

### REFERENCES

1 Ferlay J, Shin H R, Bray F, Forman D, Mathers C and Parkin D M, Estimates of worldwide burden of cancer in 2008: GLOBOCAN 2008. Int J Cancer 2010.
2 Sociaty A C, http://www.cancer.org/research/cancerfastsstatistics/cancerfactsfigures2014.
3 Institute N C, http://www.cancer.qov/cancertopics/types/liver.
4 Hepatitis, http://www.hivandhepatitis.com/hep_c/news/2010/0615_2010_b.html.
5 Yang J D and Roberts L R, Hepatocellular carcinoma: A global view. Nat Rev Gastroenterol Hepatol 2010; 7: 448-58.
6 EI-Serag H B and Rudolph K L, Hepatocellular carcinoma: epidemiology and molecular carcinogenesis. Gastroenterology 2007; 132: 2557-76.
7 Nordenstedt H, White D L and EI-Serag H B, The changing pattern of epidemiology in hepatocellular carcinoma. Dig Liver Dis 2010; 42 Suppl 3: S206-14.
8 Liou I and Kowdley K V, Natural history of nonalcoholic steatohepatitis. J Clin Gastroenterol 2006; 40 Suppl 1: S11-6.
9 Clark J M, The epidemiology of nonalcoholic fatty liver disease in adults. J Clin Gastroenterol 2006; 40 Suppl 1: S5-10.
10 Clavien P A, Lesurtel M, Bossuyt P M, Gores G J, Langer B and Perrier A, Recommendations for liver transplantation for hepatocellular carcinoma: an international consensus conference report. Lancet Oncol 2012; 13: ell-22.
11 Hanish S I and Knechtle S J, Liver transplantation for the treatment of hepatocellular carcinoma. Oncology (Williston Park) 2011; 25: 752-7.
12 Memon K, Lewandowski R J, Riaz A and Salem R, Yttrium 90 microspheres for the treatment of hepatocellular carcinoma. Recent Results Cancer Res 2013; 190: 207-24.
13 Cheng A L, Kang Y K, Chen Z, Tsao C J, Qin S, Kim J S, Luo R, Feng J, Ye S, Yang T S, et al., Efficacy and safety of sorafenib in patients in the Asia-Pacific region with advanced hepatocellular carcinoma: a phase III randomised, double-blind, placebo-controlled trial. Lancet Oncol 2009; 10: 25-34.
14 Llovet J M, Ricci S, Mazzaferro V, Hilgard P, Gane E, Blanc J F, de Oliveira A C, Santoro A, Raoul J L, Forner A, et al., Sorafenib in advanced hepatocellular carcinoma. N Engl J Med 2008; 359: 378-90.
15 Razumilava N and Gores G J, Sorafenib for HCC: a pragmatic perspective. Oncology (Williston Park) 2011; 25: 300, 302.
16 Abdel-Rahman O and Fouad M, Risk of cardiovascular toxicities in patients with solid tumors treated with sunitinib, axitinib, cediranib or regorafenib: An updated systematic review and comparative meta-analysis. Crit Rev Oncol Hematol 2014; 92: 194-207.
17 Worns M A, Koch S, Niederle I M, Marquardt J U, Nguyen-Tat M, Gamstatter T, Schuchmann M, Schulze-Bergkamen H, Galle P R and Weinmann A, The impact of patient and tumour baseline characteristics on the overall survival of patients with advanced hepatocellular carcinoma treated with sorafenib. Dig Liver Dis 2013; 45: 408-13.
18 Norton J T, Witschi M A, Luong L, Kawamura A, Ghosh S, Stack M S, Sim E, Avram M J, Appella D H and Huang S, Synthesis and anticancer activities of 6-amino amonafide derivatives. Anticancer Drugs 2008; 19: 23-36.
19 Liu Y, Norton J T, Witschi M A, Xu Q, Lou G, Wang C, Appella D H, Chen Z and Huang S, Methoxyethylamino-numonafide Is an Efficacious and Minimally Toxic Amonafide Derivative in Murine Models of Human Cancer. Neoplasia 2011; 13: 453-60.
20 Ingrassia L, Lefranc F, Kiss R and Mijatovic T, Naphthalimides and azonafides as promising anti-cancer agents. Curr Med Chem 2009; 16: 1192-213.
21 Costanza M E, Weiss R B, Henderson I C, Norton L, Berry D A, Cirrincione C, Winer E, Wood W C, Frei E, 3rd, McIntyre O R, et al., Safety and efficacy of using a single agent or a phase II agent before instituting standard combination chemotherapy in previously untreated metastatic breast cancer patients: report of a randomized study—Cancer and Leukemia Group B 8642. J Clin Oncol 1999; 17: 1397-406.
22 Paull K D, Shoemaker R H, Hodes L, Monks A, Scudiero D A, Rubinstein L, Plowman J and Boyd M R, Display and analysis of patterns of differential activity of drugs against human tumor cell lines: development of mean graph and COMPARE algorithm. J Natl Cancer Inst 1989; 81: 1088-92.
23 Savaraj N, Liang J, Lu K, Feun L G and Hsu T C, Genotoxicity of [1H]benz[de]isoquinoline-1,3[2H]dione,5 amino-2-,[2-dimethylamino) ethyl] (BIDA) in human lymphocytes. Cancer Invest 1989; 7: 117-21.
24 Shiraha H, Yamamoto K and Namba M, Human hepatocyte carcinogenesis (review). Int J Oncol 2013; 42: 1133-8.
25 Deng K, Guo X, Wang H and Xia J, The IncRNA-MYC regulatory network in cancer. Tumour Biol 2014; 35: 9497-503.
26 Gabay M, Li Y and Felsher D W, MYC activation is a hallmark of cancer initiation and maintenance. Cold Spring Harb Perspect Med 2014; 4.
27 Sewastianik T, Prochorec-Sobieszek M, Chapuy B and Juszczynski P, MYC deregulation in lymphoid tumors: molecular mechanisms, clinical consequences and therapeutic implications. Biochim Biophys Acta 2014; 1846: 457-67.
28 Choi H N, Bae J S, Jamiyandorj U, Noh S J, Park H S, Jang K Y, Chung M J, Kang M J, Lee D G and Moon W S, Expression and role of SIRT1 in hepatocellular carcinoma. Oncol Rep 2011; 26: 503-10.
29 Knight J R and Milner J, SIRT1, metabolism and cancer. Curr Opin Oncol 2012; 24: 68-75.
30 Su J C, Chiang H C, Tseng P H, Tai W T, Hsu C Y, Li Y S, Huang J W, Ko C H, Lin M W, Chu P Y, et al., RFX-1-dependent activation of SHP-1 inhibits STAT3 signaling in hepatocellular carcinoma cells. Carcinogenesis 2014; 35: 2807-14.
31 Wu C, Sun M, Liu L and Zhou G W, The function of the protein tyrosine phosphatase SHP-1 in cancer. Gene 2003; 306: 1-12.
32 Liu L, Cao Y, Chen C, Zhang X, McNabola A, Wilkie D, Wilhelm S, Lynch M and Carter C, Sorafenib blocks the RAF/MEK/ERK pathway, inhibits tumor angiogenesis, and induces tumor cell apoptosis in hepatocellular carcinoma model PLC/PRF/5. Cancer Res 2006; 66: 11851-8.
33 Cervello M, Bachvarov D, Lampiasi N, Cusimano A, Azzolina A, McCubrey J A and Montalto G, Molecular mechanisms of sorafenib action in liver cancer cells. Cell Cycle 2012; 11: 2843-55.
34 Innocenti F, Iyer L and Ratain M J, Pharmacogenetics of anticancer agents: lessons from amonafide and irinotecan. Drug Metab Dispos 2001; 29: 596-600.
35 Cao Z, Fan-Minogue H, Bellovin D I, Yevtodiyenko A, Arzeno J, Yang Q, Gambhir S S and Felsher D W, MYC phosphorylation, activation, and tumorigenic potential in hepatocellular carcinoma are regulated by HMG-CoA reductase. Cancer Res 2011; 71: 2286-97.
36 Wu G, Lu X, Wang Y, He H, Meng X, Xia S, Zhen K and Liu Y, Epigenetic high regulation of ATAD2 regulates the Hh pathway in human hepatocellular carcinoma. Int J Oncol 2014; 45: 351-61.
37 Dunn L L, Buckle A M, Cooke J P and Ng M K, The emerging role of the thioredoxin system in angiogenesis. Arterioscler Thromb Vasc Biol 2010; 30: 2089-98.
38 Watanabe R, Nakamura H, Masutani H and Yodoi J, Anti-oxidative, anti-cancer and anti-inflammatory actions by thioredoxin 1 and thioredoxin-binding protein-2. Pharmacol Ther 2010; 127: 261-70.
39 Morrison J A, Pike L A, Sams S B, Sharma V, Zhou Q, Severson J J, Tan A C, Wood W M and Haugen B R, Thioredoxin interacting protein (TXNIP) is a novel tumor suppressor in thyroid cancer. Mol Cancer 2014; 13: 62.
40 Peterson C W and Ayer D E, An extended Myc network contributes to glucose homeostasis in cancer and diabetes. Front Biosci (Landmark Ed) 2011; 16: 2206-23.
41 Zhou J, Yu Q and Chng W J, TXNIP (VDUP-1, TBP-2): a major redox regulator commonly suppressed in cancer by epigenetic mechanisms. Int J Biochem Cell Biol 2011; 43: 1668-73.
42 Lane T, Flam B, Lockey R and Kolliputi N, TXNIP shuttling: missing link between oxidative stress and inflammasome activation. Front Physiol 2013; 4: 50.
43 Lu J and Holmgren A, The thioredoxin antioxidant system. Free Radic Biol Med 2014; 66: 75-87.
44 Chutkow W A, Patwari P, Yoshioka J and Lee R T, Thioredoxin-interacting protein (Txnip) is a critical regulator of hepatic glucose production. J Biol Chem 2008; 283: 2397-406.
45 Suh H W, Yun S, Song H, Jung H, Park Y J, Kim T D, Yoon S R and Choi I, TXNIP interacts with hEcd to increase p53 stability and activity. Biochem Biophys Res Commun 2013; 438: 264-9.
46 Jung H, Kim M J, Kim D O, Kim W S, Yoon S J, Park Y J, Yoon S R, Kim T D, Suh H W, Yun S, et al., TXNIP maintains the hematopoietic cell pool by switching the function of p53 under oxidative stress. Cell Metab 2013; 18: 75-85.
47 Jin H O, Seo S K, Kim Y S, Woo S H, Lee K H, Yi J Y, Lee S J, Choe T B, Lee J H, An S, et al., TXNIP potentiates Redd1-induced mTOR suppression through stabilization of Redd1. Oncogene 2011; 30: 3792-801.
48 Yamaguchi F, Hirata Y, Akram H, Kamitori K, Dong Y, Sui L and Tokuda M, FOXO/TXNIP pathway is involved in the suppression of hepatocellular carcinoma growth by glutamate antagonist MK-801. BMC Cancer 2013; 13: 468.
49 Rohner N, Jarosz D F, Kowalko J E, Yoshizawa M, Jeffery W R, Borowsky R L, Lindquist S and Tabin C J, Cryptic variation in morphological evolution: HSP90 as a capacitor for loss of eyes in cavefish. Science 2013; 342: 1372-5.
50 Schmitz R, Ceribelli M, Pittaluga S, Wright G and Staudt L M, Oncogenic mechanisms in Burkitt lymphoma. Cold Spring Harb Perspect Med 2014; 4.
51 Rhee Y H, Jeong S J, Lee H J, Lee H J, Koh W, Jung J H, Kim S H and Sung-Hoon K, Inhibition of STAT3 signaling and induction of SHP1 mediate antiangiogenic and antitumor activities of ergosterol peroxide in U266 multiple myeloma cells. BMC Cancer 2012; 12: 28.
52 Bergeron S, Dubois M J, Bellmann K, Schwab M, Larochelle N, Nalbantoglu J and Marette A, Inhibition of the protein tyrosine phosphatase SHP-1 increases glucose uptake in skeletal muscle cells by augmenting insulin receptor signaling and GLUT4 expression. Endocrinology 2011; 152: 4581-8.
53 Dubois M J, Bergeron S, Kim H J, Dombrowski L, Perreault M, Fournes B, Faure R, Olivier M, Beauchemin N, Shulman G I, et al., The SHP-1 protein tyrosine phosphatase negatively modulates glucose homeostasis. Nat Med 2006; 12: 549-56.
54 Simmons G E, Jr., Pruitt W M and Pruitt K, Diverse roles of SIRT1 in cancer biology and lipid metabolism. Int J Mol Sci 2015; 16: 950-65.
55 Liu T F and McCall C E, Deacetylation by SIRT1 Reprograms Inflammation and Cancer. Genes Cancer 2013; 4: 135-47.
56 Proteinatlas, http://www.proteinatlas.org/ENSG00000096717-SIRT1/cancer.
57 Mao B, Zhao G, Lv X, Chen H Z, Xue Z, Yang B, Liu D P and Liang C C, Sirt1 deacetylates c-Myc and promotes c-Myc/Max association. Int J Biochem Cell Biol 2011; 43: 1573-81.
58 Avram M J, Spyker D A, Henthorn T K and Cassella J V, The pharmacokinetics and bioavailability of prochlorperazine delivered as a thermally generated aerosol in a single breath to volunteers. Clin Pharmacol Ther 2009; 85: 71-7.

What is claimed is:

1. A method of treating, ameliorating, or preventing hepatocellular carcinoma in a patient comprising administering to said patient a therapeutically effective amount of 6-methoxyethylamino-numonafide (MEAN): 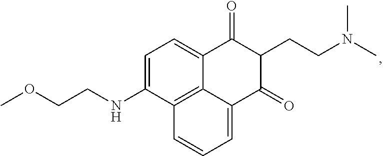
including pharmaceutically acceptable salts, solvates, and/or prodrugs thereof.

2. The method of claim 1, wherein the administering results in decreased c-myc expression.

3. The method of claim 1, wherein the administering results in increased SHP-1 expression.

4. The method of claim 1, wherein the administering results in increased TXNIP expression.

5. The method of claim 1, further comprising co-administration of a therapeutically effective amount of sorafenib 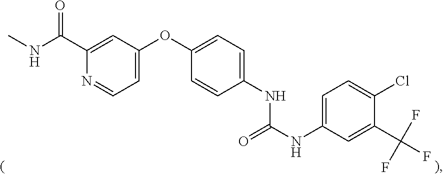 [SMILES: CNC(=O)c1cc(Oc2ccc(NC(=O)Nc3ccc(Cl)c(C(F)(F)F)c3)cc2)ccn1]
including pharmaceutically acceptable salts, solvates, and/or prodrugs thereof.

6. The method of claim 1, wherein said patient is a human patient.

7. A method of treating, ameliorating, or preventing hepatocellular carcinoma in a patient comprising administering to said patient a therapeutically effective amount of a pharmaceutical composition comprising a therapeutically effective amount of 6-methoxyethylamino-numonafide (MEAN): 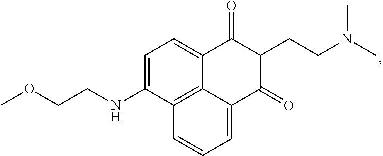
including pharmaceutically acceptable salts, solvates, and/or prodrugs thereof.

8. The method of claim 7, wherein the administering results in decreased c-myc expression.

9. The method of claim 7, wherein the administering results in increased SHP-1 expression.

10. The method of claim 7, wherein the administering results in increased TXNIP expression.

11. The method of claim 7, wherein said patient is a human patient.

12. The method of claim 7, further comprising co-administration of a pharmaceutical composition comprising a therapeutically effective amount of sorafenib 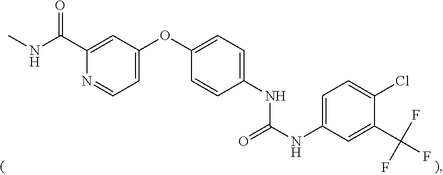 [SMILES: CNC(=O)c1cc(Oc2ccc(NC(=O)Nc3ccc(Cl)c(C(F)(F)F)c3)cc2)ccn1]
including pharmaceutically acceptable salts, solvates, and/or prodrugs thereof.

13. A kit comprising a composition comprising 6-methoxyethylamino-numonafide (MEAN): 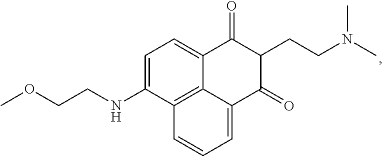
and instructions for administering such a composition to a patient having hepatocellular carcinoma.

14. The kit of claim 13, wherein the composition is a pharmaceutical composition.

15. The kit of claim 13, further comprising a composition comprising sorafenib

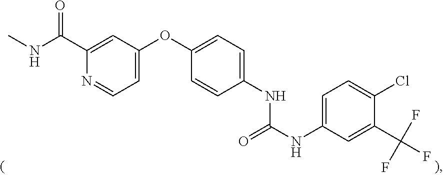 [SMILES: CNC(=O)c1cc(Oc2ccc(NC(=O)Nc3ccc(Cl)c(C(F)(F)F)c3)cc2)ccn1]

16. The kit of claim 15, wherein the composition comprising sorafenib is a pharmaceutical composition.
